(19) United States
(12) Patent Application Publication
(10) Pub. No.: US 2002/0123058 A1
(43) Pub. Date: Sep. 5, 2002

(54) Method for ultra-high resolution mapping of genes and determination of genetic networks among genes underlying phenotypic traits

(76) Inventors: David W. Threadgill, Chapel Hill, NC (US); Robert W. Williams, Memphis, TN (US)

Correspondence Address: JENKINS & WILSON, PA, 3100 TOWER BLVD, SUITE 1400, DURHAM, NC, 27707, US

(21) Appl. No.: 09/998,058

(22) Filed: Nov. 30, 2001

Related U.S. Application Data
(60) Provisional application No. 60/250,706, filed on Dec. 1, 2000.

(51) Int. Cl. C12Q 1/68; A01K 67/027

(52) U.S. Cl. 435/6; 800/18

(57) ABSTRACT

A novel population for genetic mapping, methods for generating the disclosed population, and methods for using the disclosed population for efficient identification of genetic loci that modulate a phenotype. Also provided are methods for identifying interactions between genetic loci and non-genetic factors that modulate a phenotype. Further provided are methods for defining epistatic relationships among genes gene networks and non-genetic factors.

### CROSS REFERENCE TO RELATED APPLICATIONS

This application is based on and claims priority to U.S. Provisional Application Serial No. 60/250,706, filed Dec. 1, 2000, the entire contents of which are herein incorporated by reference.

### TECHNICAL FIELD

The present invention pertains to a novel population for genetic mapping, methods for generating the disclosed population, and methods for using the disclosed population for efficient identification of genetic loci that modulate a phenotype. Methods are also provided for identifying an interaction between genetic loci and non-genetic factors that modulate a phenotype. Further, methods are provided for defining epistatic relationships among genes or gene networks.

Table of Abbreviations

| | |
|---|---|
| 129 - | a mouse non-recombinant inbred line |
| A - | a mouse non-recombinant inbred line |
| AXB - | a mouse RI line |
| BALB/c - | a mouse non-recombinant inbred line |
| BXA - | a mouse RI line |
| BXD - | a mouse RI line |
| C57BL/6 - | a mouse non-recombinant inbred line |
| cM - | centimorgan |
| CXB - | a mouse RI line |
| DBA/2 - | a mouse non-recombinant inbred line |
| F1 - | first filial generation |
| F2 - | progeny produced by F1 parents |
| LOD - | logarithmic odds ratio |
| LRS - | likelihood ratio statistic |
| PCR - | polymerase chain reaction |
| QTL - | quantitative trait loci |
| RFLP - | restriction fragment length |
| | polymorphism |
| RI - | recombinant inbred |
| RIST - | Recombinant Inbred Segregation Test |
| RIX - | recombinant inbred hybrid produced |
| | by intercrossing RI lines |
| SNP - | single nucleotide polymorphism |
| SSLP - | short sequence length polymorphism |
| STRP - | short tandem repeat polymorphism |

### BACKGROUND ART

The potential ability to link a trait with the genes responsible for that trait provides opportunities for new diagnostics and treatments of genetic diseases. A current challenge in methods for gene mapping is identification of complex trait loci. Routine approaches have largely failed to identify genes for complex traits and often yield disparate findings. A major obstacle lies in achieving fine mapping resolution for the detected loci.

The genetic component of many complex traits is oligogenic or polygenic, each contributory gene having a modest effect. Although the individual effect of a single gene is small, interactions with other genes and/or environments substantially contribute to the manifestation of the trait. A failure to recognize and accommodate such interactions in genetic mapping approaches can mask the effects of individual genes. Existing strategies for gene mapping generally rely on large sample sizes that achieve low genetic noise or renewable inbred populations that achieve low environmental noise, but not both. Additionally, such populations do not reliably consider phenotypes modulated by both genetic loci and non-genetic factors.

Genetic mapping analysis comprises progressive resolution of target gene detection. A typical experiment employs linkage analysis of the target loci and genetic polymorphisms. An initial genome-wide scan is generally conducted using a two-generation backcross or intercross. The progeny are genotyped to define an approximately 20 cM interval, or about one-quarter of a mouse chromosome, in which the target loci resides. A map location is then estimated using interval mapping or variations of the technique, wherein linkage analysis is performed using additional genetic polymorphisms within the interval. Further evaluation of candidate genes within a small chromosomal interval is variably difficult depending on the resolution of the mapping and the power to detect genetic loci with small effects. Current approaches to fine resolution mapping include selective phenotyping (SP), recombinant progeny testing (RPT), interval-specific congenic strains (ISCS), and recombinant inbred segregation test (RIST), each described immediately below. A significant difference among the approaches is the design of the population being mapped. See Darvasi (1998) Nat Genetics 18(1):19-24.

Selective phenotyping involves production of a large F2 or backcross population, and individuals that are recombinant within a previously defined interval are selected for phenotypic analysis. A smaller interval is therein defined, and an optional subsequent round of analysis evaluates recombinants within the smaller interval. This approach is effective for trait loci with large effects, when a resolution not beyond about 5 cM is necessary, and when extensive resources to accommodate many crosses are available. An F2 mapping population offers limited interpretation, however, because it is non-regenerative and does not consider phenotypes modulated by environmental variation.

Recombinant progeny testing entails genetic mapping using a population generated by backcrosses. Individuals carrying a distinguishable recombinant chromosome at a region of interest are crossed to one of the parental strains to determine the location of the target loci relative to the recombination point. This is an efficient method for identifying loci having dominant effects, but fails to consider modulation by genetic variants found in a diverse population.

Another genetic mapping approach employs interval-specific congenic stains, which are strains that differ from one another only with respect to a small chromosomal segment. According to this method, recombinant progeny are first selected based on a recombination event within a known interval. Secondarily, recombinant individuals are backcrossed to a parent strain several times to eliminate alleles from the donor parent strain at all other loci affecting the trait. Progeny are then intercrossed, and homozygotes for the recombinant haplotype are selected to establish interval-specific congenic strains. See e.g., Darvasi (1997) Mamm Genome 8:163-167 and Demant et al. (1986) Immunogenetics 24:416-422. This strategy enables fine mapping of moderate or small effects. However, since the target loci are not selected from a segregating population, fixed genetic and environmental effects can confuse results.

The Recombinant Inbred Segregation Test (RIST) is also capable of detecting small effects, although requires the availability of a recombinant inbred strain with a recombination in the region of interest. To produce an RIST population, recombinant inbred lines are backcrossed to each parental inbred line. The resulting F1 population is both backcrossed and intercrossed. Target loci will segregate in the F2 or backcross population since it was previously mapped to the vicinity of a recombination site in the recombinant inbred line. The location of target loci is described relative to the recombination site. A panel of RI strains is considered, and the collective results map the loci to a small interval defined by the relevant recombination site of each recombinant inbred line. After backcrossing or intercrossing the F1 population, each animal has a unique recombinant genotype that cannot be reproduced by natural or assisted mating.

Ideally, genetic mapping studies employ a population having maximal genetic diversity, such as a natural population, and the same population can be evaluated in diverse environmental settings. Natural populations encompass individuals that are genetically diverse and each genotype is unique. However, environmental effects cannot be efficiently controlled since the unique genotypes cannot be reproduced by natural or assisted mating.

Thus, a current and long-felt need in the field is the development of more mapping strategies that are more efficient and offer improved power to detect complex trait loci and improved mapping resolution. The present invention discloses a novel mapping population for ultra-high resolution genetic mapping of any trait, and thus addresses the current and long-felt need in the art for the same.

### SUMMARY OF THE INVENTION

The present invention provides a method for identifying a genetic locus that modulates a phenotype. According to the method, a renewable population of genetically diverse individuals is provided for genetic mapping. The genomes of individuals within the renewable population of genetically diverse individuals that display a phenotype are mapped, thereby identifying a genetic locus that modulates the phenotype.

The present invention further provides a method for identifying an interaction between a genetic locus and a non-genetic factor, wherein the interaction modulates a phenotype. According to this method, a renewable population of genetically diverse individuals is generated, and a non-genetic factor is provided to the renewable population. The genomes of individuals that display the phenotype are mapped whereby a genetic locus that interacts with the non-genetic factor to modulate the phenotype is identified. Preferably, the renewable population of genetically diverse individuals is generated by a method of the present invention. The disclosed method for identifying an interaction between a genetic locus and a non-genetic factor also encompasses interactions among two or more loci or non-genetic factors, wherein the interaction modulates a phenotype.

The invention further provides a method for identifying an epistatic interaction between loci that modulate a phenotype. The method comprises providing a first renewable population of genetically diverse individuals. Individuals within the first renewable population that display a phenotype are identified, and genetic mapping of such individuals identifies a genetic locus that modulates the phenotype. A second renewable population of genetically diverse individuals is generated, wherein the first genetic locus is held constant. Individuals within the second renewable population of genetically diverse individuals are identified. Genetic mapping of such individuals identifies a second genetic locus that epistatically interacts with the first genetic locus to modulate the phenotype.

The methods of the present invention can be used for mapping genetic loci of any population of diploid, tetraploid, or polyploid individuals. An individual of a preferred population is an animal or a plant, or cell derived there from. More preferably, the animal is a mammal, even more preferably the mammal is a rodent, still more preferably the rodent is a mouse.

In accordance with the methods of the present invention, a renewable population of genetically diverse individuals can comprise: (a) individuals produced by intercrossing recombinant inbred lines; (b) individuals produced by backcrossing recombinant inbred lines; (c) a cloned population of genetically diverse individuals; or (d) a panel of cell lines derived from genetically diverse individuals.

In one embodiment of the invention, the renewable population of genetically diverse individuals is derived by intercrossing recombinant inbred lines. In the case of intercrosses, preferably all possible reciprocal pair wise combinations of recombinant inbred lines are considered, such that a population of n recombinant inbred lines produces a renewable population of n(n-1) individuals.

In another embodiment of the invention, the renewable population of genetically diverse individuals is produced by cloning recombinant individuals. In still another embodiment of the invention, the renewable population of genetically diverse individuals comprises a panel of cell lines derived from genetically diverse individuals. Preferably, the cloned population or the panel of cell lines is derived from recombinant inbred line intercrosses, recombinant inbred line backcrosses, an F2 population, or a natural population.

In each of the described embodiments of the invention, the recombinant inbred lines preferably comprise less than about 500 lines, and more preferably less than about 100 lines. Representative recombinant inbred lines include but are not limited to members of the mouse lines AXB, BXA, CXB, and BXD.

The recombinant inbred lines can be produced by intercrossing at least 3 non-recombinant parent lines, preferably at least 4 non-recombinant parent lines, and more preferably at least 8 non-recombinant parent lines. Representative non-recombinant parent lines include but are not limited to the mouse lines C57BL/6, BALB/c, A, 129, and DBA/2.

Preferred phenotypes that can be mapped using the methods of the present invention include but are not limited to a visible phenotype, a physiological phenotype, a behavioral phenotype, a susceptibility phenotype, a cellular phenotype, a molecular phenotype, and combinations thereof. Preferred molecular phenotypes include but are not limited to a level of gene expression, a splice selection, a level of protein, a protein type, a protein modification, a level of lipid, a lipid type, a lipid modification, a level of carbohydrate, a carbohydrate type, a carbohydrate modification, and combinations thereof. A phenotype can be further characterized as modulated by a non-genetic factor, by an interaction between two or more non-genetic factors, or by an interaction between a genetic locus and a non-genetic factor. A preferred non-genetic factor is an environmental condition or exposure to a drug.

The present invention further provides methods for generating a renewable population of genetically diverse individuals. According to the method, the renewable population is produced by: (a) intercrossing recombinant inbred lines; (b) backcrossing recombinant inbred lines; (c) cloning a population of genetically diverse individuals; (d) or generating a panel of cell lines derived from genetically diverse individuals. Also provided is a renewable population of genetically diverse individuals produced by the disclosed methods.

The present invention also provides methods for generating recombinant inbred parent lines. According to the method, three or more non-recombinant inbred lines are intercrossed to produce recombinant hybrids. Preferably, at least four non-recombinant inbred parent lines are used, and more preferably at least eight non-recombinant inbred parent lines are used. The recombinant hybrids are intercrossed one or more generations to produce a population of genetically diverse recombinant individuals. Each genetically diverse recombinant individual is then inbred to produce a recombinant inbred line. The present invention also provides a recombinant inbred line produced by the disclosed methods.

An object of the present invention is to provide a novel population of diverse individuals and methods of using the same for ultra-high resolution genetic mapping of any selected trait. This object has been met in whole or in part by the present invention.

Some of the objects of the invention having been stated hereinabove, other objects will become evident as the description proceeds when taken in connection with the accompanying Drawings and Examples as best described herein below.

### BRIEF DESCRIPTION OF THE DRAWINGS

FIG. 1 depicts a prototypical approach for producing a renewable population of genetically diverse individuals using intercrosses of recombinant inbred lines. Non-recombinant parent lines are crossed to produce recombinant progeny, which are then inbred. The resulting population comprises a multiplicity, n, of recombinant inbred parents (e.g., recombinant inbred lines). The recombinant inbred parents are then intercrossed to produce n(n-1) recombinant and genetically diverse individuals (e.g., RIX hybrids). The population of recombinant and genetically diverse individuals can be regenerated by repeating intercrosses among the recombinant inbred parents.

FIG. 2 presents a mathematical representation of the power of genetic mapping using a renewable population of genetically diverse individuals. The mapping population of the present invention comprises RIX hybrids produced by RI intercrosses. This population minimizes genetic and environmental noise, and thereby combines the advantages of RI and F2 mapping populations. y, trait value of individual; a, mean trait value of population; b, gene strength or allele substitution effect; x, target gene genotype; bx, effect of target gene on trait value; bᵢxᵢ, effect of non-target gene i.

FIG. 3 is a graphical illustration of the power to detect a target gene using a renewable population of genetically diverse individuals (▪) or using recombinant inbred populations (▴) having a same number of individuals. In this simulation, the phenotype has a high environmental noise and a fixed background genetic noise representing 13% (arrow) of the total phenotypic variance contributed by a single secondary locus. When a target gene accounts for 13% of the total phenotypic variance, the power to detect the target gene is about five times greater using a renewable population of genetically diverse individuals as compared to a recombinant inbred population.

FIG. 4 is a grid representation of the RIX population used for QTL analysis (described in Example 2). Thirteen independent CXB RI lines are designated CXB1, CXB2, CXB3, CXB4, CXB5, CXB6, CXB7, CXB8, CXB9, CXB10, CXB11, CXB12, and CXB13. CXB recombinant inbred lines were intercrossed to produce CXB recombinant inbred hybrids (CXB RIX hybrids). For each intercross, the paternal genotype is indicated on the x-axis (labeled “Father”), and the maternal genotype is indicated on the y-axis (labeled “Mother”). CXB RIX hybrids produced by the intercrosses are represented by the rectangular blocks within the grid, the position of each block indicative of a particular paternal genotype and a particular maternal genotype. The number of recombinant inbred hybrids used for QTL analysis of body weight (Example 3) is listed in each block. QTL analysis of body weight was also performed using the CXB RI parents BALB/cByJ, CB6ByF1, and C57BL/6ByJ (Example 3). The number of CXB RI parents included in the analysis is listed at the top adjacent to each CXB RI parent genotype.

FIG. 5 is a chromosomal map depicting a locus on mouse chromosome 4 (D5Mit372) that shows suggestive linkage to body weight. The locus was identified by QTL analysis of CXB RI parents as described in Example 3. Vertical lines, LOD score; black plot, likelihood ratio statistic; gray plot, additive effect.

FIGS. 6A-6C are chromosomal maps depicting loci on mouse chromosomes 4, 6, and 12 that are linked to body weight. The loci were identified by QTL analysis of RIX hybrids as described in Example 3.

FIG. 6A is a chromosomal map depicting a locus on mouse chromosome 4 that is significantly linked to body weight. The locus was identified by QTL analysis of RIX hybrids as described in Example 3 and corresponds to the locus identified by QTL analysis of RI parents (D5Mit372). See FIG. 5. Vertical lines, LOD score; black plot, likelihood ratio statistic; gray plot, additive effect; (●) plot, dominance effect.

FIG. 6B is a chromosomal map depicting two loci on mouse chromosome 6 that are significantly linked to body weight. The loci were identified by QTL analysis of RIX hybrids as described in Example 3. Vertical lines, LOD score; black plot, likelihood ratio statistic; gray plot, additive effect; (●) plot, dominance effect.

FIG. 6C is a chromosomal map depicting a locus on mouse chromosome 12 that are significantly linked to body weight. The locus was identified by QTL analysis of RIX hybrids as described in Example 3. Vertical lines, LOD score; black plot, likelihood ratio statistic; gray plot, additive effect; (●) plot, dominance effect.

FIG. 7 is a chromosomal map depicting a locus on mouse chromosome 11 that shows suggestive linkage to brain weight. The locus was identified by QTL analysis of CXB RI parents as described in Example 4. Vertical lines, LOD score; black plot, likelihood ratio statistic; gray plot, additive effect.

FIGS. 8A-8B are chromosomal maps depicting loci on mouse chromosomes 5 and 11 that are linked to brain weight. The loci were identified by QTL analysis of RIX hybrids as described in Example 4.

FIG. 8A is a chromosomal map depicting a locus on mouse chromosome 5 that shows suggestive linkage to brain weight. The locus was identified by QTL analysis of RIX hybrids as described in Example 4. Green vertical lines, LOD score; black plot, likelihood ratio statistic; red plot, additive effect; blue plot, dominance effect.

FIG. 8B is a chromosomal map depicting a locus on mouse chromosome 11 that is significantly linked to brain weight. The locus was identified by QTL analysis of RIX hybrids as described in Example 4 and corresponds to the locus identified by QTL analysis of RI parents. See FIG. 7. Green vertical lines, LOD score; black plot, likelihood ratio statistic; red plot, additive effect; blue plot, dominance effect.

FIG. 9 is a grid representation of hippocampal weight in CXB RIX hybrids. Thirteen independent CXB RI lines are designated CXB1, CXB2, CXB3, CXB4, CXB5, CXB6, CXB7, CXB8, CXB9, CXB10, CXB11, CXB12, and CXB13. CXB recombinant inbred lines were intercrossed to produce CXB recombinant inbred hybrids (CXB RIX). For each intercross, the paternal genotype is indicated on the x-axis (labeled “Dad”), and the maternal genotype is indicated on the y-axis (labeled “Mom”). RIX hybrids produced by the intercrosses are represented by the rectangular blocks within the grid, the position of each block indicative of a particular paternal genotype and a particular maternal genotype. The mean hippocampal weight for RIX hybrids of a particular genotype is indicated in the blocks. For comparison, QTL analysis of hippocampal weight was also performed using the CXB RI parents BALB/cByJ and C57BL/6ByJ. The hippocampal weight of each CXB RI parent genotype included in the analysis is listed at the top adjacent to each genotype. Boxed blocks identify pairs of reciprocal RIX hybrids with significantly different hippocampal weights.

### DETAILED DESCRIPTION OF THE INVENTION

The present invention discloses methods for generating a renewable population of genetically diverse individuals and methods for using such a population for efficient mapping of genetic loci that modulate a phenotype. Also disclosed are methods for identifying interactions among genetic and non-genetic factors, or among gene networks, wherein the interaction modulates a phenotype. The invention further discloses a method for cell-based gene mapping using cell lines derived from a renewable population of genetically diverse individuals or any relevant diverse cell population.

I. Definitions

While the following terms are believed to be well understood by one of ordinary skill in the art, the following definitions are set forth to facilitate explanation of the invention.

I.A. Populations

The term “population” refers to any group of individuals.

The term “individual” refers to any diploid or polyploid organism, or a cell derived there from.

The term “inbred” describes substantially isogenic individuals produced by crossing of closely related individuals. The term “inbreeding” refers to repeated crossing of closely related individuals.

The term “isogenic lines” refers to a population of individuals that are genetically identical at all loci.

The term “nonrecombinant inbred line” refers to an inbred line wherein individuals are genetically identical at all loci.

The term “recombinant inbred line”, abbreviated herein as “RI”, refers to an inbred line derived from two unrelated inbred parent lines. An individual RI line has a characteristic combination of genes with a different pattern of alternative alleles at multiple loci.

The term “congenic line” and “congenic strain” each refer to strains that differ from one another only with respect to a small chromosomal segment. A congenic line is a recombinant inbred line wherein alternative alleles all reside in a limited chromosomal interval. Recombinant congenic strains are produced by a series of backcrosses to a parent line followed by inbreeding (Allard et al., 1966; Demant & Hart, 1986). An interval specific congenic strain is recombinant at a specific 1 cM interval (Darvasi, 1997).

The terms “chromosomal substitution strain” and “consomic strain” each refer to an inbred line that is identical to a first inbred line, host line A, with the exception that a single chromosome is replaced by the corresponding chromosome from a second inbred line, donor line B. Chromosome substitution strains are recombinant inbred lines wherein alternative alleles all reside on the single substituted chromosome. See Nadeau et al. (2000) Nat Genet 24:221-225.

The term “intercross” refers to the mating of individuals that are each heterozygous at a selected genetic loci. The term “intercross” encompasses “advanced intercross”, meaning crosses between subsequent generations of intercrossed offspring. The terms “intercross” and “advanced intercross” are understood to include mating or assisted fertilization to produce intercross progeny. Preferred methods for assisted fertilization or reproduction include but are not limited to cloning, in vitro fertilization, or inter-cytoplasmic sperm injection. Methods for assisted fertilization are well known in the art as disclosed in Nakagata (2000) Mamm Genome 11:572-576; Thornton et al. (1999) Mamm Genome 10:987-992; Loutradis et al. (2000) Ann NY Acad Sci 900:325-335; and in U.S. Pat. Nos. 5,453,366, 5,541,081, 5,849,713.

The abbreviation “RIX” refers to intercrosses between recombinant inbred lines.

The term “backcross” refers to a cross between an offspring and one of its parents or an individual genetically identical to one of its parents. The term “backcross” encompasses “advanced backcross”, meaning crosses between a backcross progeny and an inbred progenitor from a prior generation or an individual genetically identical to an inbred progenitors from a prior generation. The terms “backcross” or “advance backcross” are understood to include mating or assisted fertilization to generate backcross progeny. Preferred methods for assisted fertilization or reproduction include but are not limited to cloning, in vitro fertilization, or inter-cytoplasmic sperm injection. Methods for assisted fertilization are well known in the art (Thornton et al., 1999; Loutradis et al., 2000; Nakagata, 2000) U.S. Pat. Nos. 5,453,366, 5,541,081, 5,849,713)

The term “natural population” refers to a group of individuals that exists in nature and generally lacks intervention comprising experimental selection of mating pairs.

The term “F2 population” refers to the progeny produced by intercrossing, assisted fertilization, or self-fertilization of F1 individuals. The term “F1 individuals” refers to the first filial generation.

I.B. Genetic Mapping

The term “mapping”, “genetic mapping”, “mapping of the genome”, or “genotyping” each refer to a method for describing a position of a genetic locus in terms of recombination frequency with a genetic polymorphism. The results of a mapping method are described in map units or Morgans.

The term “polymorphism” refers to the occurrence of two or more genetically determined alternative sequences or alleles in a population. An allelic difference can be as small as one base pair.

The term “Morgan” or “map unit” each refer to a unit for expressing the relative distance between genes on a chromosome. One Morgan unit (M) indicates a recombination frequency of 100%. A centimorgan (cM) indicates a recombination frequency of 1%. The term “recombination frequency” refers to the number of recombinants divided by the total number of progeny.

The term “power” as used herein refers to the probability of detecting or mapping a genetic locus. Power is preferably 80%, more preferably 90%, even more preferably 95%, and even more preferably 99%. The power of detection is correlated with target gene strength, and is optimal when genetic noise and environmental noise in the mapping population is low. Conversely, the power of detection is diminished by genetic noise and environmental noise.

The term “target gene” in the context of genetic mapping refers to the gene residing at a genetic locus that contributes to a phenotype.

The term “strength” and “target gene strength” each refer to the percent contribution of a single gene to a phenotype. Gene strength correlates with ease of genetically detecting the genetic locus. Relatively strong target genes are easily detected. Genes with relatively weak effects contribute to complex traits, and are often masked by environmental noise.

The term “genetic noise” or “genetic background” or “residual genotype” as used herein each refer to a level of genetic variation. In a genetic mapping experiment, genetic noise is inversely correlated with genetic diversity. For example, genetic noise is significant in a recombinant inbred population due to the limited number of unique genotypes. A level of genetic noise can be described by the equation:

genetic noise=Σbᵢxᵢ,

Wherein b represents gene strength or allele substitution effect, x represents genotype, and i represents a number of non-target genes. Thus, genetic noise represents a sum of allele substitution effects at all non-target loci contributing to a phenotype. Optimally, the genetic noise should approach zero for maximum sensitivity of gene mapping.

The term “environmental noise” or environmental background” as used herein each refer to a level of environmental variation. In a genetic mapping experiment, environmental noise is inversely correlated with experimental replication of identical genotypes. For example, environmental noise is significant when all individuals are unique, as in an F2 population. Optimally, the environmental noise should approach zero for maximum sensitivity of gene mapping.

The term “epistatic interaction” refers to a nonreciprocal interaction between nonallelic genetic loci, between gene networks, or between one or more genetic loci and one or more non-genetic factors. Thus, the term “epistatic” encompasses both linear and non-linear interactions.

The term “about”, as used herein when referring to a measurable value such as a position of a locus (e.g., in cM), target gene strength, power, etc., is meant to encompass variations of ±20% or ±10%, more preferably ±5%, even more preferably ±1%, and still more preferably ±0.1% from the specified value, as such variations are appropriate to perform the disclosed method.

I.C. Traits

The term “phenotype” or “trait” each refer to any observable property of an organism, produced by the interaction of the genotype of the organism and the environment. A phenotype can encompass variable expressivity and penetrance of the phenotype. Exemplary phenotypes include but are not limited to a visible phenotype, a physiological phenotype, a behavioral phenotype, a susceptibility phenotype, a cellular phenotype, a molecular phenotype, and combinations thereof.

The term “expressivity” refers to the severity or intensity of a phenotype displayed by individuals of a specified genotype when examined under a define set of environmental conditions.

The term “penetrance” refers to a proportion of individuals of a specific genotype that display the selected genotype when examined under a defined set of environmental conditions.

The term “molecular phenotype” refers to a detectable feature of molecules in a cell or organism. Exemplary molecular phenotypes include but are not limited to a level of gene expression, a splice selection, a level of protein, a protein type, a protein modification, a level of lipid, a lipid type, a lipid modification, a level of carbohydrate, a carbohydrate type, a carbohydrate modification, and combinations thereof. Methods for observing, detecting, and quantitating molecular phenotypes are well known to one skilled in the art. See Ausubel (ed.) (1995) Short Protocols in Molecular Biology, 3rd ed. Wiley, New York; Bodanszky (1993) Principles of Peptide Synthesis, 2nd rev. ed. Springer-Verlag Berlin/New York; Harlow & Lane (1988) Antibodies: A Laboratory Manual. Cold Spring Harbor Laboratory Press, Cold Spring Harbor, N.Y.; Innis (1 990) PCR Protocols: A Guide to Methods and Applications. Academic Press, San Diego; Koduri & Poola (2001) Steroids 66:17-23; Landegren et al. (1988) Science 242:229-237; Regan et al. (2000) Anal Biochem 286:265-276;

Sambrook et al. (1989) Molecular Cloning: A Laboratory Manual. Cold Spring Harbor Laboratory Press, Cold Spring Harbor, N.Y.; Silhavy et al. (1984) Experiments with Gene Fusions. Cold Spring Harbor Laboratory, Cold Spring Harbor, N.Y.; and U.S. Pat. Nos. 6,096,555; 5,958,624; and 5,629,158.

The term “susceptibility phenotype” refers to an increased capacity or risk for displaying a phenotype.

The term “complex trait” as used herein refers to a trait that is not inherited as predicted by classical Mendelian genetics. A complex trait results from the interaction of multiple genes, each gene contributing to the phenotype. Complex traits can be continuous or show threshold penetrance.

The term “oligogenic trait” refers to a complex trait determined by a few genes, each having a moderate effect.

The term “polygenic trait” refers to a complex trait determined by many genes, each having a moderate effect.

The term “gene network” and “genetic network” each refer to a set of two or more genes that function cooperatively to generate a phenotype. A gene network comprises complex trait loci.

The term “quantitative trait” is a complex trait that can be assessed quantitatively. Quantitation entails measurement of a trait across a continuous distribution of values.

The term “effect”, in the context of gene traits, refers to the contribution of an individual gene in the expression of a phenotype. A gene effect can be described qualitatively, e.g. a large or small effect, or can be quantitated as percent contribution of an individual genetic locus to a phenotype.

The term “modulate” in the context of a phenotype, refers to the action of a genetic or non-genetic factor to contribute to the phenotype. Modulation can promote or detract from expressivity or penetrance of the phenotype. Alternatively or in addition, modulation can add or subtract specific features of a phenotype. A modulatory contribution can be dramatic or subtle, the only requirement being that it is ultimately detectable.

The term “segregate” in the context of genetics, refers to allele sorting among progeny of a genetic cross, wherein individuals with a phenotype are distinguishable. The term “segregating trait” refers to a phenotype that is distinguishable in a subset of progeny resulting from a genetic cross.

I.D. Non-Genetic Factors

The term “non-genetic factor” refers to any condition exclusive of genotype that modulates a phenotype. A non-genetic factor is any element of the environment, including but not limited to a habitat condition, a level of activity or exercise, diet, a drug treatment, and combinations thereof.

The term “drug” is any substance that affects a physical, physiological, behavioral, mental, cellular, or molecular function of a living organism. A drug can be a chemical compound, a protein, a peptide, a lipid, a carbohydrate, a nucleic acid, any other bioactive agent, and combinations thereof.

II. Generation of a Renewable Population of Genetically Diverse Individuals

The phrase “renewable population of genetically diverse individuals” refers to a population that can be faithfully regenerated and comprises a limited repertoire of possible genotypes, although individuals within the population are genetically diverse.

The term “individual” as used herein refers to an organism or a cell derived there from. The population can comprise any diploid, tetraploid, or polyploid individual. Preferably, an individual of the renewable population is an animal or a plant. A preferred animal is a mammal, more preferably a rodent, even more preferably a mouse.

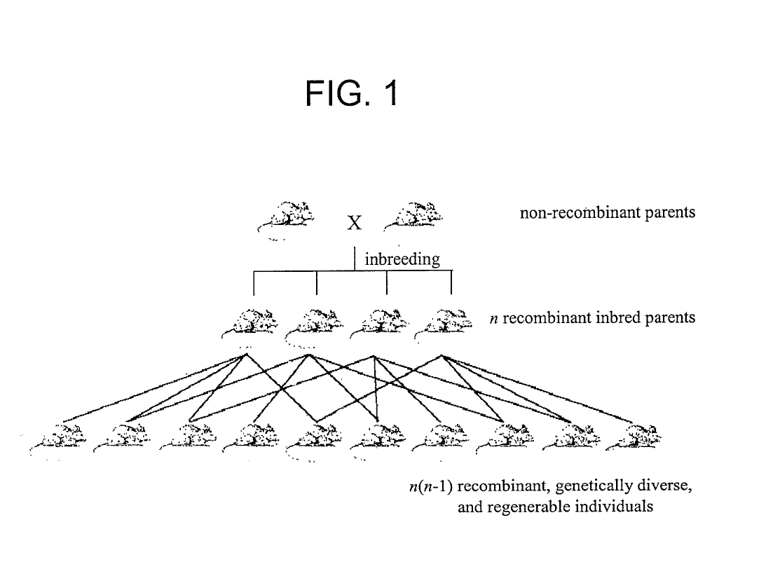

In one embodiment of the invention, a renewable population of genetically diverse individuals is generated by intercrossing recombinant inbred lines. Preferably, the recombinant inbred lines comprise a number of lines, n, that is used to generate a renewable population of genetically diverse individuals comprising n(n-1) individuals, representing all possible reciprocal pair wise combinations of recombinant inbred lines (FIG. 1).

In another embodiment of the invention, a renewable population of genetically diverse individuals is generated by backcrossing or assisting fertilization of recombinant inbred lines and parental non-recombinant inbred lines.

In another embodiment of the invention, the renewable population of genetically diverse individuals is produced by cloning genetically diverse individuals. The genetically diverse individuals include but are not limited to individuals of a population produced by recombinant inbred line intercrosses, a population produced by recombinant inbred line backcrosses, an F2 population, or a natural population. Methods for cloning are known to one skilled in the art. See U.S. Pat. Nos. 5,994,619, 6,011,197, and 6,107543; Sun & Moor (1995) Curr Top Dev Biol 30:147-176; Cibelli et al. (1998) Science 280:1256-1258; Wilmut et al. (1997) Nature 385:810-813; Wakayama et al. (2000) Nature 407:318-319; Wakayama et al. (1999) Proc Natl Acad Sci US A 96:14984-14989; Wakayama et al. (1998) Nature 394:369-374; and DiBerardino (1997) Genomic Potential of Differentiated Cells. Columbia University Press, New York.

In another embodiment of the present invention, the renewable population of genetically diverse individuals comprises a panel of cell lines derived from genetically diverse individuals. Any population of genetically diverse individuals can be used, including but not limited to a population produced by recombinant inbred line intercrosses, a population produced by recombinant inbred line backcrosses, an F2 population, or a natural population. In contrast to an F2 population comprising organisms, a population comprising cell lines derived there from offers increased control of environmental noise. Methods for producing cell lines are well known in the art as disclosed in U.S. Pat. Nos. 4,707,448; 5,643,782; and 5,114,847; and PCT International Publication No. WO 98/58050. Preferably, a panel of 25 different cell lines is used for gene mapping, and more preferably at least 100 different cell lines are used.

In each of the described embodiments, the recombinant inbred lines preferably comprise less than about 500 lines, and more preferably less than about 100 lines. However, the mapping methods of the present invention are not limited by the number of recombinant lines used, and can therefore employ greater than 500 lines, for example 1000 RI lines, or 2000 RI lines, or 5000 RI lines, or any number of RI lines that permit identification of QTL loci in accordance with the disclosed methods. Representative types of recombinant inbred lines include but are not limited to congenic lines and chromosome substitution strains. Exemplary recombinant inbred lines include but are not limited to the members of mouse lines AXB, BXA, CXB, and BXD (Lyon et al., 1996).

The recombinant inbred lines are derived from at least three non-recombinant inbred lines, more preferably from at least four non-recombinant inbred lines, and still more preferably from at least eight non-recombinant inbred lines. Exemplary non-recombinant parent lines include but are not limited to the mouse lines C57BL/6, BALB/c, A, 129, and DBA/2 (Lyon et al., 1996).

The present invention also provides methods for generating recombinant inbred parent lines. According to the method, non-recombinant inbred lines are intercrossed to produce recombinant hybrids. The recombinant hybrids are intercrossed one or more generations to produce a population of genetically diverse recombinant individuals. Each recombinant individual is inbred to produce a recombinant inbred line. Preferably, at least four non-recombinant inbred parent lines are used, and more preferably at least eight non-recombinant inbred parent lines are used. The method reduces the level of homozygous genome segments in early stages of generating a recombinant inbred line. The present invention also provides a recombinant inbred line produced by the disclosed methods. Such recombinant lines are characterized as having higher productive or detectable recombination frequencies than in currently available lines.

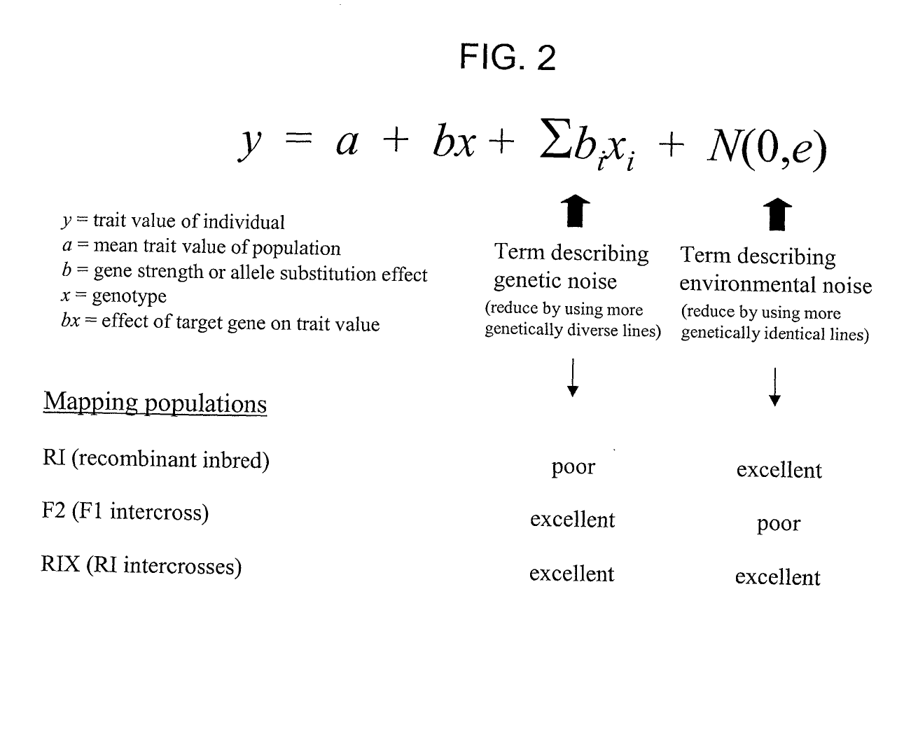

A novel aspect of the disclosed mapping approach lies in the features of the mapping population. In contrast to existing populations for genetic mapping, a renewable population of genetically diverse individuals, or a panel of cell lines representing individuals derived from a diverse population, is characterized by minimal genetic noise as well as environmental noise (FIG. 2). Recombinant inbred lines are a commonly used mapping population that has substantially low environmental noise, but power of detection is hindered by poor genetic diversity. Conversely, the relatively high genetic diversity among individuals of an F2 population offers low genetic noise, but locus detection is difficult due to high environmental noise.

A representative population of RIX hybrids that can be used in accordance with the methods of the present invention is described in Example 2. Preferably, a population of RIX hybrids used for QTL comprises a sufficient number of genetically diverse RIX hybrid individuals to optimize QTL detection. For example, a population of RIX hybrids can comprise a population derived by performing all possible pair wise crosses between about 100 recombinant inbred parents, or between about 500 recombinant inbred parents.

III. Identification of a Genetic Locus that Modulates a Phenotype

The present invention also discloses a method for identifying a genetic locus that modulates a phenotype using a renewable population of genetically diverse individuals. The disclosed method also encompasses identifying of two or more genetic loci that modulate a phenotype.

Representative methods for identifying genetic loci linked to body weight and brain weight are described in Examples 3 and 4, respectively. Mapping methods that employ RIX hybrids show improved sensitivity of detection of linked loci when compared to methods that employ RI parents.

Preferred phenotypes include but are not limited to a visible phenotype, a physiological phenotype, a behavioral phenotype, a susceptibility phenotype, a cellular phenotype, a molecular phenotype, and combinations thereof. Preferred molecular phenotypes include but are not limited to a level of gene expression, a splice selection, a level of protein, a protein type, a protein modification, a level of lipid, a lipid type, a lipid modification, a level of carbohydrate, a carbohydrate type, a carbohydrate modification, and combinations thereof. A phenotype can be further characterized as modulated by a non-genetic factor, an interaction between two or more non-genetic factors, an interaction between a genetic locus and a non-genetic factor, or an interaction between two or more genetic loci and non-genetic factors. A preferred non-genetic factor is an environmental condition or exposure to a drug.

Techniques for genetic mapping are well known to one skilled in the art, including linkage analysis (e.g., (Wells & Brown, 2000)), linkage disequilibrium analysis (Kruglyak, 1999), restriction landmark genomic scanning (RLGS) (Akiyoshi et al., 2000), and radiation hybrid mapping (Schuler et al., 1996; Van Etten et al., 1999). Any suitable mapping technique can be used, and it will be appreciated by one of skill in the art that no particular choice is essential to or a limitation of the present invention.

A preferred method for genetic mapping is linkage analysis whereby a phenotype is correlated with one or more detectable polymorphisms including but not limited to restriction fragment length polymorphisms (RFLPs) (Lander & Botstein, 1989), short tandem repeat polymorphisms (STRPs), short sequence length polymorphisms (SSLPs) (Dietrich et al., 1996), microsatellite markers (Schalkwyk et al., 1999), and single nucleotide polymorphisms (SNPs) (Brookes, 1999).

A preferred technique for linkage analysis is detection of SNPs. The density of SNP markers in a mammalian genome is estimated to be about 1 SNP per 1 kb of sequence. See Collins et al. (1998) Genome Res 8:1229-1231. Several approaches can be used for typing SNPs, including homogenous hybridization assays (Livak et al., 1995), oligonucleotide ligation assays (Chen et al., 1998), matrix-assisted laser desorption time-of-flight mass spectrometry (MALDI-TOF) (Kwok, 1998; Ross et al., 1998), high performance liquid chromatography (HPLC) (Schriml et al., 2000), fluorescence polarization (Chen et al., 1999), array-based technologies (Cronin et al., 1996; Hacia et al., 1996; Pastinen et al., 1997; Gentalen & Chee, 1999; Sapolsky et al., 1999), pyrominisequencing (Nyren et al., 1993), and invader methods (Griffin et al., 1999; Lyamichev et al., 1999). See also Landegren et al. (1998) Genome Res 8:769-776.

Preferred methods for SNP detection are array-based oligonucleotide hybridization and minisequencing, described further herein below, as these techniques are amenable to high-throughput and multiplex formats. Oligonucleotide microarrays or chips can be manufactured by photolithographic synthesis of oligonucleotides onto glass slides using for example, the AFFYMETRIX® system (Affymax Corporation of Greenford Middlesex, Great Britain) See Fodor et al. (1991) Science 251:767-773 and U.S. Pat. No. 5,445,934. Alternatively, oligonucleotide microarrays can be produced by gridding oligonucleotides robotically onto the surface of a slide or other solid support (Schena et al., 1996), or by using an inkjet type technology to deliver oligonucleotides to a solid support (U.S. Pat. No. 5,965,352). By either method, a particular SNP is determined by a position of an oligonucleotide having an SNP in the array. To detect a SNP using a hybridization assay, genomic fragments of a test genome are amplified by PCR and labeled such that the fragment is detectable. A SNP of the test genome is determined by the formation of a detectable heteroduplex structure at an identified position in the array. To perform minisequencing reactions on chips, genomic fragments of a test genome are amplified using PCR and hybridized to an oligonucleotide microarray. Primer extension reactions including labeled nucleotides are performed on the hybridized oligonucleotide array. A SNP of the test genome is identified as a successful primer extension reaction assayed by detecting the labeled nucleotides. Alternatively, the SNP can be detected by amplification on the solid support without prior PCR.

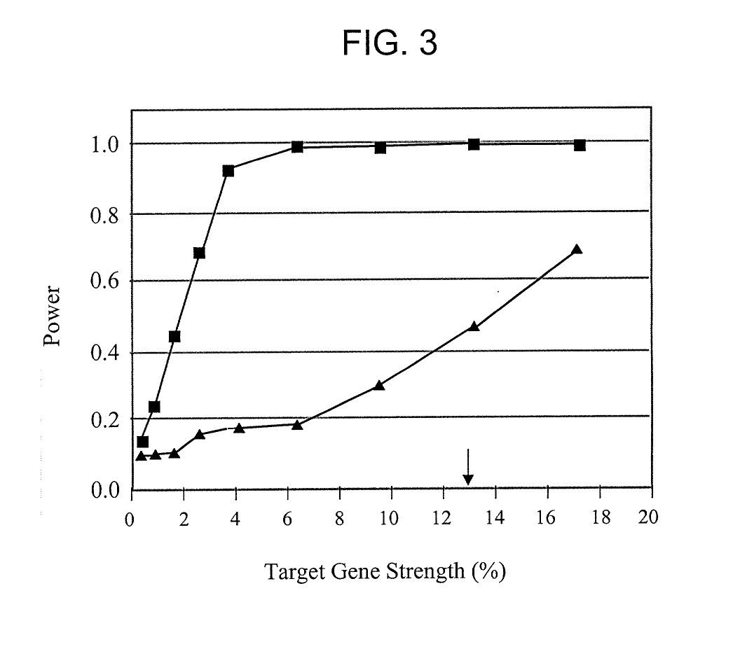

Detection of SNPs in a renewable population of genetically diverse individuals enables a genetic locus to potentially be mapped within a 1 cM interval. This ultra fine resolution mapping defines an approximately 10⁶ base pair region comprising about 50 genes. The methods of the present invention enable enhanced power of detection compared to current mapping approaches (FIG. 3).

A simulated QTL mapping analysis is described in Example 1. The simulation reveals a significantly increased power of detecting a QTL in a population of RIX hybrids when compared to a power of detecting a QTL in an RI population. Representative QTL mapping analyses to identify loci that control body weight and brain weight are described in Examples 3 and 4, respectively. Representative methods for subsequent QTL validation are described in Example 5. QTL mapping using RIX hybrids can also be used to determine parental effect loci, as described in Example 6.

Regional cloning based on the genetic map position can be used to clone genes residing at the locus using methods known in the art. Alternatively, an integrated gene and physical map framework can be used to reference one or more genes at the mapping position. See Klysik et al. (1999) Genomics 62:123-128.

IV. Interactions Between Genetic Loci and Non-Genetic Factors

The present invention further provides a method for identifying an interaction between a genetic locus and a non-genetic factor, wherein the interaction modulates a phenotype. According to this method, a renewable population of genetically diverse individuals is generated, and a non-genetic factor is provided to the renewable population. The genomes of individuals that display the phenotype are mapped so that a genetic locus that interacts with the non-genetic factor to modulate the phenotype is identified. Preferably, the renewable population of genetically diverse individuals is generated by a method of the present invention, as disclosed herein above.

The disclosed method for identifying an interaction between a genetic locus and a non-genetic factor also encompasses interactions among two or more genetic loci or non-genetic factors, wherein the interaction modulates a phenotype.

V. Epistatic Interactions

The invention further provides a method for identifying an epistatic interaction between loci that modulate a phenotype. The method comprises providing a first renewable population of genetically diverse individuals. Individuals within the first renewable population that display a phenotype are identified, and genetic mapping of such individuals identifies a genetic locus that modulates the phenotype. A second renewable population of genetically diverse individuals is generated, wherein the first genetic locus is held constant. Individuals within the second renewable population of genetically diverse individuals are identified. Genetic mapping of such individuals identifies a second genetic locus that epistatically interacts with the first genetic locus to modulate the phenotype. Epistatic interactions can also occur between genetic and non-genetic factors. Furthermore, epistatic interactions can occur between a visible phenotype, a physiological phenotype, a behavioral phenotype, a susceptibility phenotype, a cellular phenotype, a molecular phenotype, and combinations thereof.

VI. Genetic Map Database

The present invention also provides a relational database system for storing genetic mapping information in a searchable format. Relevant genetic mapping information includes but is not limited to descriptions of mapping populations, phenotypes assayed, linkage analysis data using SNPs or other polymorphisms, non-genetic factors assayed, interactions between genetic loci and non-genetic factors, epistatic relationships, and alignment of genetic and physical maps. The relational database can integrate mapping information derived from studies in related organisms, preferably experiments done in mouse and human. The relational database can also provide links to relevant resources including gene and protein databases, chemical or drug databases, medical information databases, and biological depositories. The relational database can further provide interfaces and methods for analyzing mapping data, including but not limited to statistical calculations of mapping resolution and genetic mapping simulations that assist in designing an appropriate mapping population.

VII. Summary

Summarily, the present invention provides a novel population and methods of using the same for genetic mapping. The disclosed mapping populations optimize genetic and environmental diversity, thereby enabling significant power of detection of complex traits. The present invention further provides methods for using the disclosed population to identify an interaction between a genetic locus and a non-genetic factor, wherein the interaction modulates a phenotype. Also provided are methods for determining an epistatic interaction between genes, gene networks, and non-genetic factors.

### EXAMPLES

The following Examples have been included to illustrate modes of the invention. Certain aspects of the following Examples are described in terms of techniques and procedures found or contemplated by the present co-inventors to work well in the practice of the invention. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the invention.

### Example 1

### Detection of a Target Gene

Using Recombinant Inbred Intercrosses

Fifteen (15) different recombinant inbred lines are intercrossed such that 120 of a possible 210 individual crosses are performed. Each of 120 crosses is replicated 8 times. The resulting renewable population of 960 genetically diverse individuals is used to simulate the detection of a target gene more efficiently than current methods (FIG. 3).

### Example 2

### Generation of an RIX Population

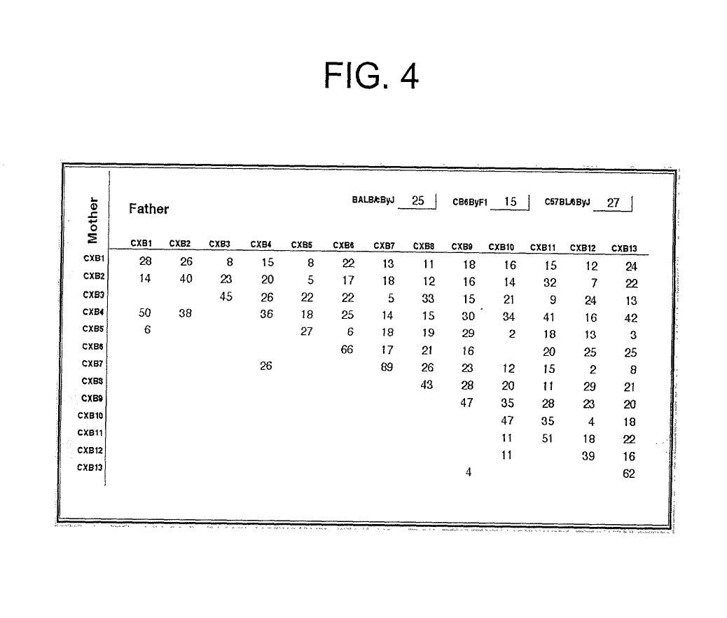

Recombinant inbred hybrids were generated using a collection of 13 independent CXB recombinant inbred lines (Bailey, 1971; Swank & Bailey, 1973; Potter et al., 1975; Hilgers et al., 1985). The CXB RI lines are derived from BALB/cByJ and C57BL/6ByJ. Pair wise crosses used to produce recombinant inbred hybrids are outlined in FIG. 4. Seventy-eight (78) non-reciprocal and 8 reciprocal recombinant inbred hybrids were generated.

The term “non-reciprocal RIX hybrid” refers to a population of hybrids produced by: (a) intercrossing male animals of a first RI line with female animals of a second RI line; or (b) intercrossing male animals of the second RI line with female animals of the first RI line.

The term “reciprocal RIX hybrid” refers to one hybrid of a pair of hybrids, wherein one of the pair of hybrids is produced by intercrossing male animals of a first RI line with female animals of a second RI line, and the other of the pair of hybrids is produced by intercrossing male animals of the second RI line with female animals of the first RI line.

### Example 3

### QTL Analysis of Body Weight

RIX hybrids, generated as described in Example 2, and RI parents were used to analyze body weight, which is a morphometric characteristic controlled by quantitative trait loci. The genotypes of the CXB RI parents are described by Williams et al. (2001) Genome Biology2:0046.1-0046.18, and the genotypes of the RIX hybrids (generated as described in Example 2) were inferred from the parental genotypes. Interval mapping and composite interval mapping of loci linked to body weight were analyzed using the Map Manager QTX program (publicly available at http://mapmgr.roswellpark.org/mmQTX.html). See Manly et al. (2001) Mamm Genome 12:930-932.

The analysis employed a free regression model, which is a conventional method in the field for assessing a likelihood that association of a quantitative trait and a gene locus is statistically significant. Briefly, according to the model, an estimate is made of the additive effect and dominance effect of an allele at each gene locus, for example the “B allele” at locus B. The magnitude of the additive effect and dominance effect, expressed as a regression coefficient, is varied freely using standard least square methods until the regression relation accounts for as much of the variance in the trait as possible. The regression coefficients represent estimates of the additive and dominance effects of the B allele. The estimates of the additive effect and dominance effect of the B allele are compared relative to zero, and the statistical significance of a potential difference from zero is estimated in consideration of sample size and the number of degrees of freedom in the regression model.

The term “additive effect” refers to an estimate of the predicted linear effect on a trait value by substituting a single copy of a first gene variant (an allele) with a second gene variant. For example, when considering two alleles of a gene, B and b, wherein the trait value of cases with the bb genotype is 100, and wherein the trait value of cases with the BB genotype is 200, the effect of substituting a single b allele with a B allele is predicted to produce an increase of 50 units. Thus, the additive effect of the B allele is described as 50 units. Conversely, the linear or additive effect of the b allele is described as −50 units.

The term “dominance effect” refers to a deviation from an expected value of a heterozygote genotype (Bb) at a single locus. For example, when considering two alleles of a gene, B and b, wherein the trait value of cases with the bb genotype is 100, and wherein the trait value of cases with the BB genotype is 200, the average trait value for cases with a Bb genotype is predicted to be 150 units. However, an observed average trait value of the Bb genotype is 175 units. Thus, in this case the dominance effect of the B allele is described as +25 units.

Simple genetic systems can be similarly modeled. For example, a strictly additive model does not incorporate a coefficient for any dominance effect of the B allele. A model can also be developed to assess multi-locus non-linear interactions (e.g., epistatic interactions), for example interactions among alleles at the B locus and those at one or more other loci.

Mapping results determined using Map Manager QTX were expressed as a likelihood ratio statistic, which was converted manually to a LOD value. The term “likelihood ratio statistic”, abbreviated herein as “LRS”, refers to a measure of the strength of statistical association between variance in the trait (e.g., differences in body weight) and genetic differences at a particular locus, or genetic differences at an interval between loci. LRS values are distributed in the same way as a chi-square distribution. High LRS values indicate that an association is unlikely to occur by random or by chance. An LRS score ≧10 is suggestive of a gene locus modulating a trait; and an LRS score ≧15 is often statistically significant.

An LRS score can be converted to a LOD score by dividing the LRS score by 4.6. The level of significance for detected loci was evaluated based on LOD standards typically used in the field. A LOD score of 2 suggests linkage, and a LOD score of 3 or greater (e.g., 3, 4, 5) indicates significant linkage. See Lander & Botstein (1989) Genetics 121:185-199.

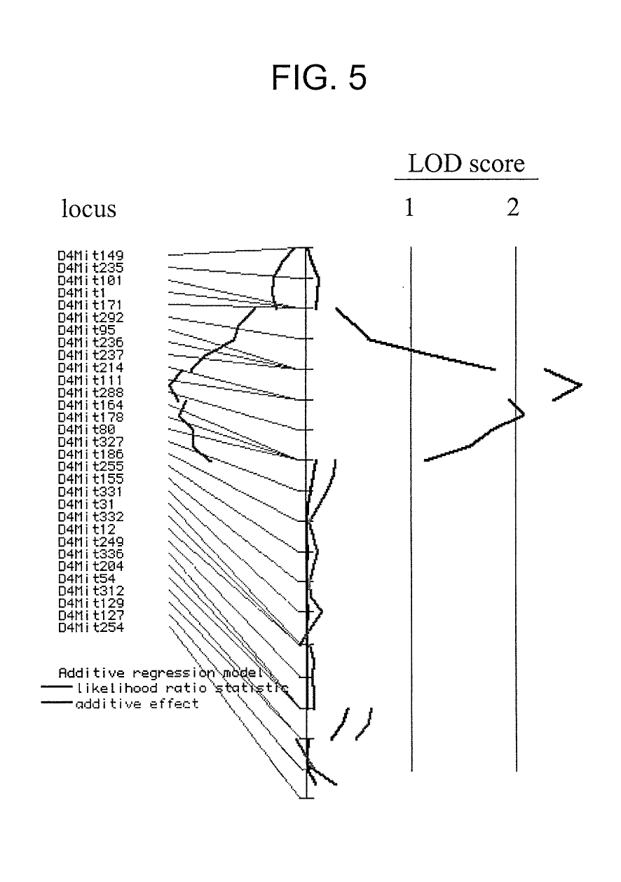

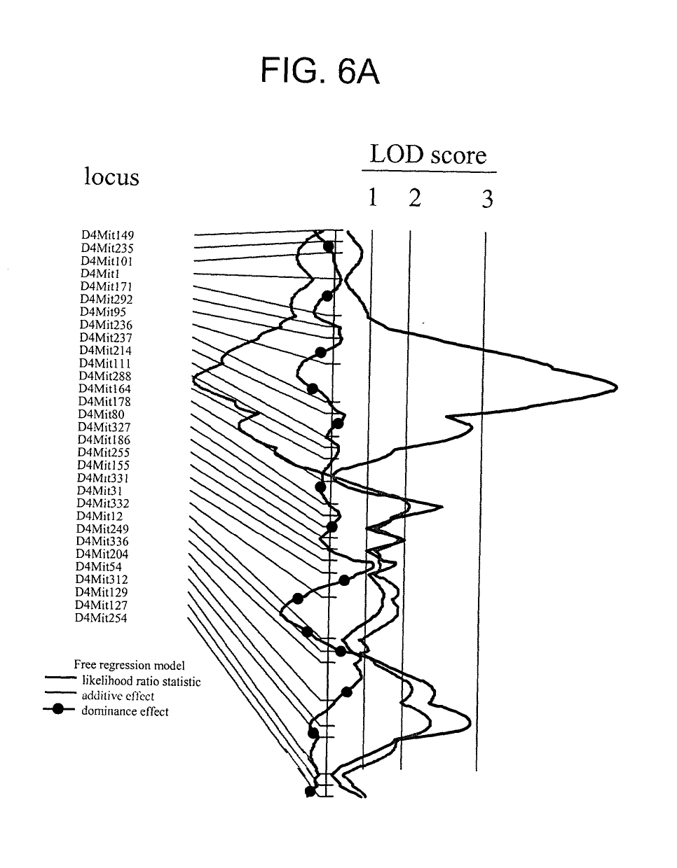

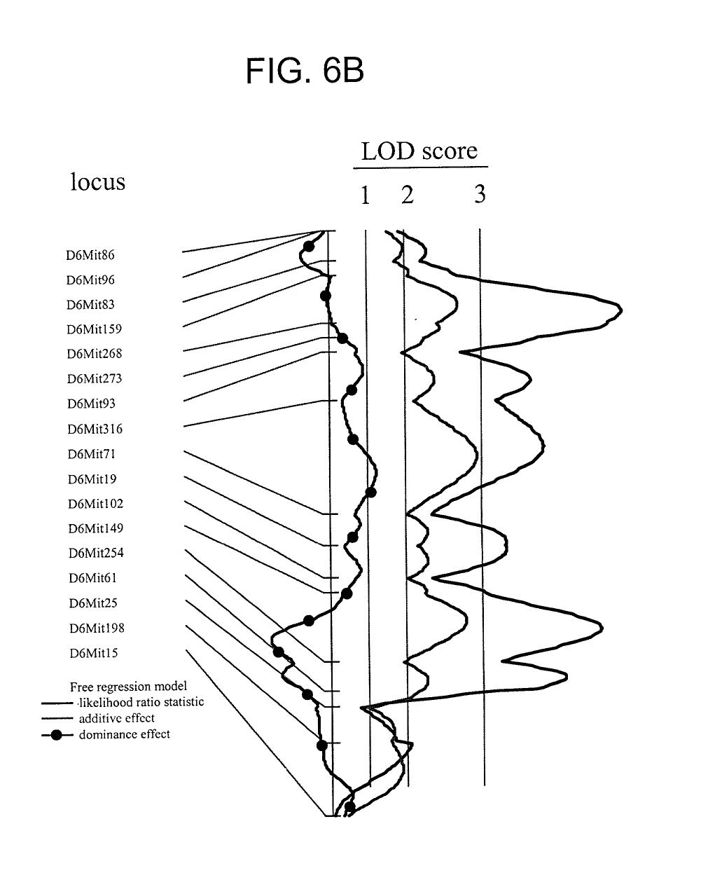

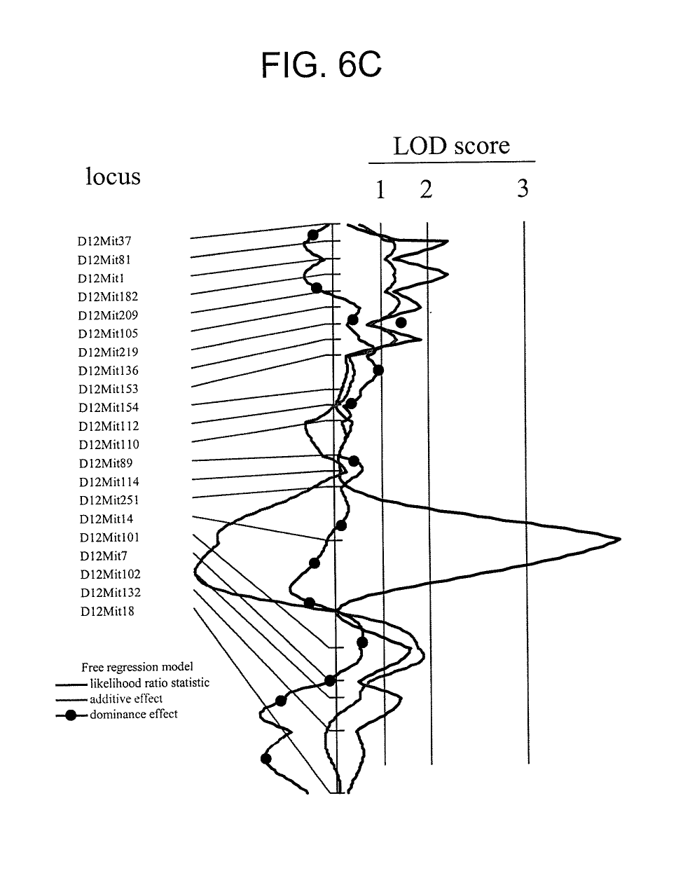

QTL analysis of body weight in a population of RIX hybrids showed improved detection and resolution of loci controlling body weight when compared to QTL analysis of the RI parents. A single suggestive locus (LOD score=2) on chromosome 4 was detected among the RI parents (FIG. 5). By contrast, QTL analysis of RIX hybrids revealed four significant loci (LOD score ≧3) and one suggestive locus (LOD score=2). One of the four significant loci corresponded to the same chromosome 4 locus detected by QTL analysis of the RI parents (FIG. 6A). Additional significant loci were located on chromosome 6 (FIG. 6D) and on chromosome 12 (FIG. 6C).

Candidate genes controlling body weight are identified within a 2-LOD confidence interval. According to standard methods in the field, a 2-LOD interval is determined by subtracting 2 LOD units from a peak LOD value. All loci greater than the subtracted result are considered within the 2-LOD interval.

For example, when mapping loci that control body weight in RI parents, a peak LOD score of about 2.5 is observed at about a position of locus D4Mit237 (FIG. 5), and any surrounding loci with a LOD score >0.5 is considered candidate QTLs. Thus, the candidate QTL is determined to reside in an about 31.4 cM genomic region of chromosome 4, which spans from about a position of locus D4Mit171 (at 6.3 cM) to about a position of locus D4Mit80(at 37.7 cM).

Mapping with RIX hybrids defines a smaller region on chromosome 4 in which a candidate QTL resides (FIG. 6A). In this case, a LOD maximum of about 5 is observed at about a position of locus D4Mit236 (12.1 cM). A 2-LOD interval centered at the maximum LOD score comprises an about 7.4 cM genomic region between about a position of locus D4Mit95 (7.5 cM) and about a position of locus D4Mit214(17.9 cM).

### Example 4

### QTL Analysis of Brain Weight

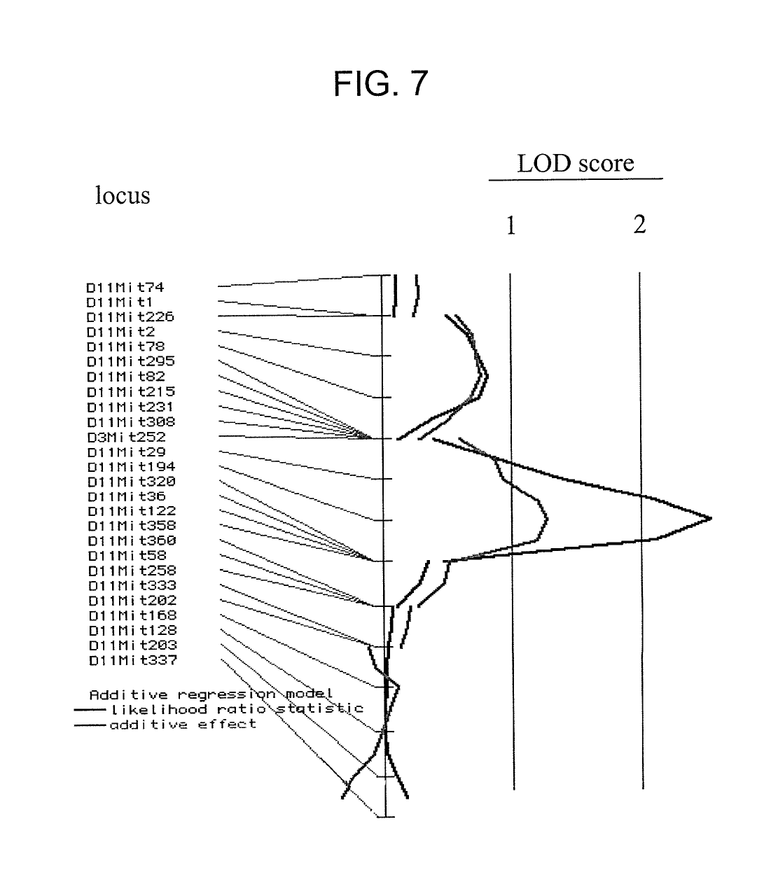

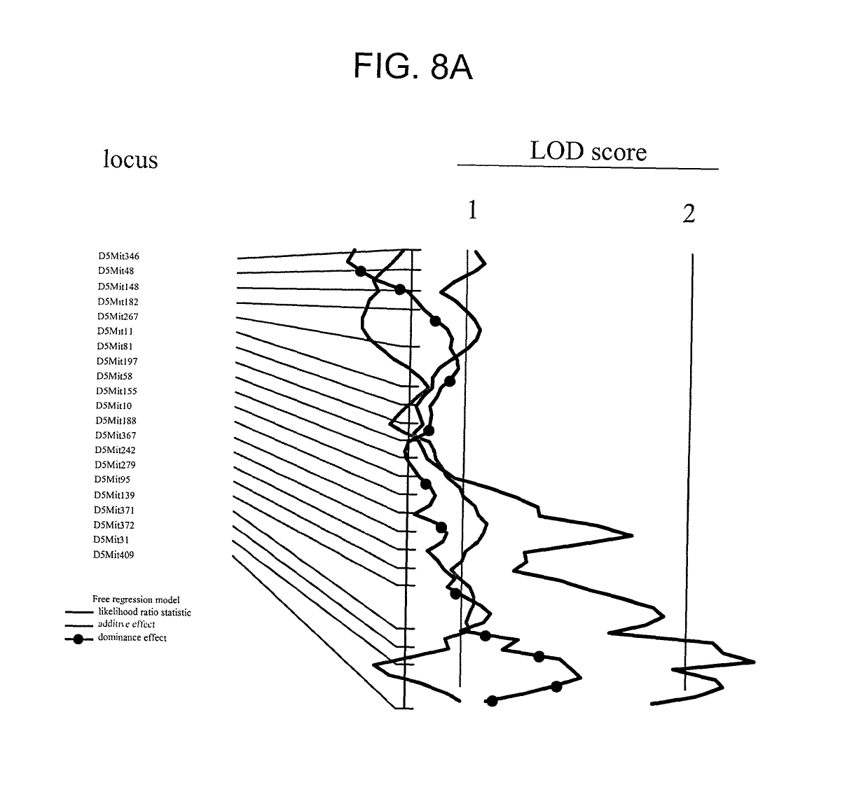

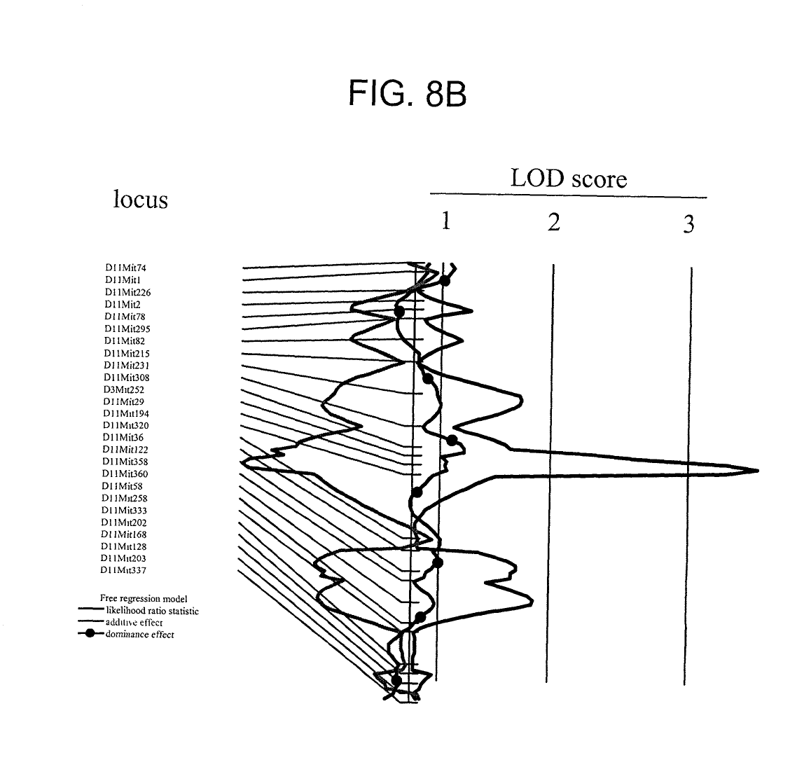

QTL analysis of brain weight was performed using RIX hybrids, prepared as described in Example 2, and RI parents. Linkage was determined as described in Example 3. Similar to the analysis of loci that control body weight (Example 3), QTL analysis of RIX hybrids also showed improved detection and resolution of loci controlling brain weight when compared to QTL analysis of the parental RI lines. A single suggestive locus (LOD score=2) on chromosome 11 was detected by QTL analysis of RI lines (FIG. 7). QTL analysis of RIX hybrids revealed one suggestive locus (LOD score=2) on chromosome 5 (FIG. 8A) and one significant locus (LOD score ≧3) on chromosome 11 (FIG. 8B). The chromosome 11 locus on chromosome 11 corresponded to the chromosome 11 locus detected by QTL analysis of RI parents (FIG. 7).

As described herein above, QTL controlling brain weight are predicted to reside within a 2-LOD interval defined by the peak LOD score. For example, mapping brain weight QTL in RI parents identifies an about 33.0 cM genomic interval spanning from about a position of locus D11Mit308(20.0 cM) to about a position of locus D11Mit122(53.0 cM) (FIG. 7). Mapping of brain weight in RIX hybrids defines a smaller interval on chromosome comprising an about 3.0 cM genomic interval, which spans from about a position of locus D11Mit29 (40.0 cM) to about a position of locus D11Mit320(43.0 cM) (FIG. 8B).

### Example 5

### Validation of Loci Identified by QTL Analysis

To validate loci detected using the RIX hybrids, a suggestive locus linked to brain weight on chromosome 5, D5Mit372, was used as a candidate locus in 300 F2 mice produced by F1 intercrosses of BALB/cByJ and C57BL/6ByJ parents. This locus was easily detected with a highly significant correlation to brain weight (p<7×10⁻⁷).

To identify QTL within a 2-LOD interval, reciprocal congenic lines can be used to further delimit the genomic region in which the QTL resides. See e.g., Darvasi (1997) Mamm Genome 8:163-167 and Demant et al. (1986) Immunogenetics 24:416-422.

Once a genomic interval is narrowed to less than about 0.3 cM, a list of genes positioned with the interval can be obtained from genome sequencing sources. (e.g., the Human Genome Project, http://www.ncbi.nlm.hih.gOV HGP/). Candidate genes are then individually tested for a role in contributing to the relevant-phenotype.

### Example 6

### Determination of Parental Effect Loci

RIX mapping can also be used to reveal parental effect loci. The term “parental effect locus” refers to a locus, wherein alleles of the locus differentially affect phenotype when inherited from the mother versus when inherited from the father.

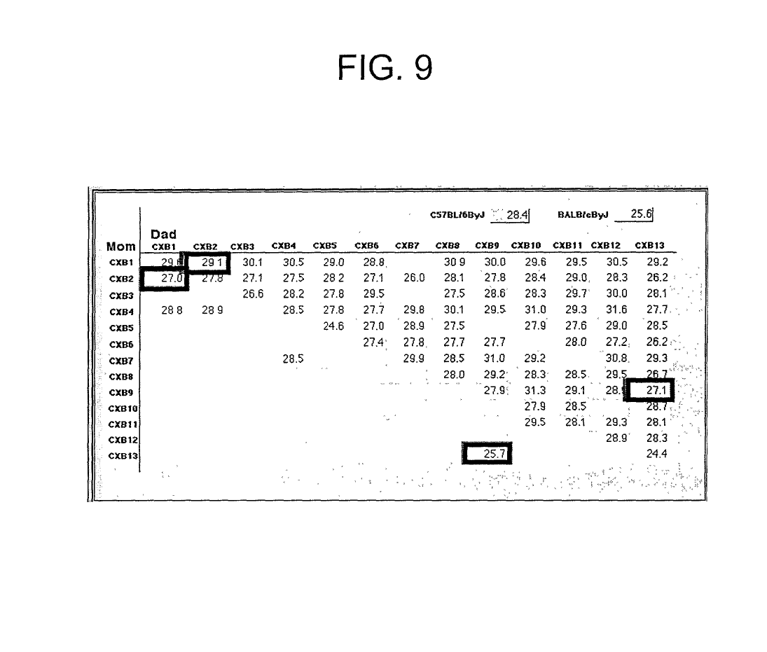

RIX hybrids produced by reciprocal crosses were analyzed for differences in hippocampal weight. Significant differences were observed for some, but not all, reciprocal crosses, indicating that the RIX hybrids can be used to identify parental effect loci (FIG. 9).

Reciprocal crosses that suggest the presence of a parental effect loci (by yielding significantly different phenotypes or phenotype measurements), can be tested further to distinguish a germ line parental effect (a true parental effect) and a maternal environment parental effect (a host parental effect). According to this approach, embryos derived from reciprocal crosses are transferred into a neutral surrogate female. If phenotypic differences are still observed, the parental effect is a germ line parental effect. If phenotypic differences are not observed, the parental effect is a host parental effect.

### References

The publications and other materials listed below and/or set forth in the text above to illuminate the background of the invention, and in particular cases, to provide additional details respecting the practice, are incorporated herein by reference. Materials used herein include but are not limited to the following listed references.

Akiyoshi S, Kanda H, Okazaki Y, Akama T, Nomura K, Hayashizaki Y & Kitagawa T (2000) A Genetic Linkage Map of the Msm Japanese Wild Mouse Strain with Restriction Landmark Genomic Scanning (RLGS). Mamm Genome 11:356-359.

Allard RW, Harding J & Wehrhahn C (1966) The Estimation and Use of Selective Values in Predicting Population Change. Heredity 21:547-563.

Ausubel F, ed (1995) Short Protocols in Molecular Biology, 3rd ed. Wiley, N.Y.

Bailey D W (1971) Recombinant-Inbred Strains. An Aid to Finding Identity, Linkage, and Function of Histocompatibility and Other Genes. Transplantation 11:325-327.

Bodanszky M (1993) Principles of Peptide Synthesis, 2nd rev. ed. Springer-Verlag, Berlin/New York.

Brookes A J (1999) The Essence of SNPs. Gene 234:177-186.

Chen X, Livak K J & Kwok P Y (1998) A Homogeneous, Ligase-Mediated DNA Diagnostic Test. Genome Res 8:549-556.

Chen X, Levine L & Kwok P Y (1999) F1 Fluorescence Polarization in Homogeneous Nucleic Acid Analysis. Genome Res 9:492-498.

Cibelli J B, Stice S L, Golueke P J, Kane J J, Jerry J, Blackwell C, Ponce de Leon F A & Robl J M (1998) Cloned Transgenic Calves Produced from Nonquiescent Fetal Fibroblasts. Science 280:1256-1258.

Collins F S, Brooks L D & Chakravarti A (1998) A DNA Polymorphism Discovery Resource for Research on Human Genetic Variation. Genome Res 8:1229-1231.

Cronin M T, Fucini R V, Kim S M, Masino R S, Wespi R M & Miyada C G (1996) Cystic Fibrosis Mutation Detection by Hybridization to Light-Generated DNA Probe Arrays. Hum Mutat 7:244-255.

Darvasi A (1997) Interval-Specific Congenic Strains (ISCS): An Experimental Design for Mapping a QTL into a 1-Centimorgan Interval. Mamm Genome 8:163-167.

Demant P & Hart A A (1986) Recombinant Congenic Strains—a New Tool for Analyzing Genetic Traits Determined by More Than One Gene. Immunogenetics 24:416-422.

DiBerardino M A (1997) Genomic Potential of Differentiated Cells. Columbia University Press, New York.

Dietrich W F, Miller J, Steen R, Merchant M A, Damron-Boles D, Husain Z, Dredge R, Daly M J, Ingalls K A, O'Connor T J & et al. (1996) A Comprehensive Genetic Map of the Mouse Genome. Nature 380:149-152.

Fodor S P, Read J L, Pirrung M C, Stryer L, Lu A T & Solas D (1991) Light-Directed, Spatially Addressable Parallel Chemical Synthesis. Science 251:767-773.

Gentalen E & Chee M (1999) A Novel Method for Determining Linkage between DNA Sequences: Hybridization to Paired Probe Arrays. NucleicAcids Res 27:1485-1491.

Griffin T J, Hall J G, Prudent J R & Smith L M (1999) Direct Genetic Analysis by Matrix-Assisted Laser Desorption/lonization Mass Spectrometry. Proc Natl Acad Sci U S A 96:6301-6306.

Hacia J G, Brody L C, Chee M S, Fodor S P & Collins F S (1996) Detection of Heterozygous Mutations in Brca1 Using High Density Oligonucleotide Arrays and Two-Colour F1 Fluorescence Analysis. Nat Genet 14:441-447.

Harlow E & Lane D (1988) Antibodies: A Laboratory Manual. Cold Spring Harbor Laboratory Press, Cold Spring Harbor, N.Y.

Hilgers J, van Nie R, Ivanyi D, Hilkens J, Michalides R, de Moes J, Poort-Keesom R, Kroezen V, von Deimling O, Kominami R & et al. (1985) Genetic Differences in BALB/C Sublines. Curr Top Microbiol Immunol 122:19-30.

Innis MA (1990) PCR Protocols: A Guide to Methods and Applications. Academic Press, San Diego.

Klysik J, Cai WW, Yang C & Bradley A (1999) An Integrated Gene and SSLP BAC Map Framework of Mouse Chromosome 11. Genomics 62:123-128.

Koduri S & Poola I (2001) Quantitation of Alternatively Spliced Estrogen Receptor Alpha mRNAs as Separate Gene Populations. Steroids 66:17-23.

Kruglyak L (1999) Prospects for Whole-Genome Linkage Disequilibrium Mapping of Common Disease Genes. Nat Genet 22:139-144.

Kwok P Y (1998) Genotyping by Mass Spectrometry Takes Flight. Nat Biotechnol 16:1314-1315.

Landegren U, Kaiser R, Caskey C T & Hood L (1988) DNA Diagnostics—Molecular Techniques and Automation. Science 242:229-237.

Lander E S & Botstein D (1989) Mapping Mendelian Factors Underlying Quantitative Traits Using RFLP Linkage Maps. Genetics 121:185-199.

Livak K J, Marmaro J & Todd J A (1995) Towards Fully Automated Genome-Wide Polymorphism Screening. Nat Genet 9:341-342.

Loutradis D, Drakakis P, Kallianidis K, Sofikitis N, Kallipolitis G, Milingos S, Makris N & Michalas S (2000) Biological Factors in Culture Media Affecting in Vitro Fertilization, Preimplantation Embryo Development, and Implantation. Ann N Y Acad Sci 900:325-335.

Lyamichev V, Mast A L, Hall J G, Prudent J R, Kaiser M W, Takova T, Kwiatkowski R W, Sander T J, de Arruda M, Arco D A, Neri B P & Brow M A (1999) Polymorphism Identification and Quantitative Detection of Genomic DNA by Invasive Cleavage of Oligonucleotide Probes. Nat Biotechnol 17:292-296.

Lyon M F, Rastan S, Brown S D M & International Committee on Standardized Genetic Nomenclature for Mice. (1996) Genetic Variants and Strains of the Laboratory Mouse, 3rd ed. Oxford University Press, Oxford; New York.

Manly K F, Cudmore Jr R H & Meer J M (2001) Map Manager QTX, Cross-Platform Software for Genetic Mapping. Mamm Genome 12:930-932.

Nadeau J H, Singer J B, Matin A & Lander E S (2000) Analysing Complex Genetic Traits with Chromosome Substitution Strains. Nat Genet24:221-225.

Nakagata N (2000) Cryopreservation of Mouse Spermatozoa. Mamm Genome 11:572-576.

Nyren P, Pettersson B & Uhlen M (1993) Solid Phase DNA Minisequencing by an Enzymatic Luminometric Inorganic Pyrophosphate Detection Assay. Anal Biochem 208:171-175.

Pastinen T, Kurg A, Metspalu A, Peltonen L & Syvanen A C (1997) Minisequencing: A Specific Tool for DNA Analysis and Diagnostics on Oligonucleotide Arrays. Genome Res 7:606-614.

Potter M, Pumphrey J G & Bailey D W (1975) Genetics of Susceptibility to Plasmacytoma Induction. I. BALB/CANN (C), C57BL/6n (B6), C57BL/KA (BK), (C Times B6)F1, (C Times BK)F1, and C Times B Recombinant-Inbred Strains. J Natl Cancer Inst 54:1413-1417.

Regan M R, Emerick M C & Agnew W S (2000) Full-Length Single-Gene cDNA Libraries: Applications in Splice Variant Analysis. Anal Biochem286:265-276.

Ross P, Hall L, Smirnov I & Haff L (1998) High Level Multiplex Genotyping by MALDI-TOF Mass Spectrometry. Nat Biotechnol 16:1347-1351.

Sambrook et al. e (1989) Molecular Cloning: A Laboratory Manual. Cold Spring Harbor Laboratory Press, Cold Spring Harbor.

Sapolsky R J, Hsie L, Berno A, Ghandour G, Mittmann M & Fan J B (1999) High-Throughput Polymorphism Screening and Genotyping with High-Density Oligonucleotide Arrays. Genet Anal 14:187-192.

Schalkwyk L C, Jung M, Daser A, Weiher M, Walter J, Himmelbauer H & Lehrach H (1999) Panel of Microsatellite Markers for Whole-Genome Scans and Radiation Hybrid Mapping and a Mouse Family Tree. Genome Res 9:878-887.

Schena M, Shalon D, Heller R, Chai A, Brown P O & Davis R W (1996) Parallel Human Genome Analysis: Microarray-Based Expression Monitoring of 1000 Genes. Proc Natl Acad Sci USA 93:10614-10619.

Schriml L M, Peterson R J, Gerrard B & Dean M (2000) Use of Denaturing HPLC to Map Human and Murine Genes and to Validate Single-Nucleotide Polymorphisms. Biotechniques 28:740-745.

Schuler G D, Boguski M S, Stewart E A, Stein L D, Gyapay G, Rice K, White R E, Rodriguez-Tome P, Aggarwal A, Bajorek E, Bentolila S, Birren B B, Butler A, Castle A B, Chiannilkulchai N, Chu A, Clee C, Cowles S, Day P J, Dibling T, Drouot N, Dunham I, Duprat S, East C, Hudson T J & et al. (1996) A Gene Map of the Human Genome. Science 274:540-546.

Silhavy T J, Berman M L, Enquist L W & Cold Spring Harbor Laboratory. (1984) Experiments with Gene Fusions. Cold Spring Harbor Laboratory, Cold Spring Harbor, N.Y.

Sun FZ & Moor RM (1995) Nuclear Transplantation in Mammalian Eggs and Embryos. Curr Top Dev Biol 30:147-176.

Swank R T & Bailey D W (1973) Recombinant Inbred Lines: Value in the Genetic Analysis of Biochemical Variants. Science 181:1249-1252.

Thornton C E, Brown S D & Glenister P H (1999) Large Numbers of Mice Established by in Vitro Fertilization with Cryopreserved Spermatozoa: Implications and Applications for Genetic Resource Banks, Mutagenesis Screens, and Mouse Backcrosses. Mamm Genome 10:987-992.

U.S. Pat. No. 4,707,448

U.S. Pat. No. 5,114,847

U.S. Pat. No. 5,445,934

U.S. Pat. No. 5,629,158

U.S. Pat. No. 5,643,782

U.S. Pat. No. 5,958,624

U.S. Pat. No. 5,965,352

U.S. Pat. No. 5,994,619

U.S. Pat. No. 6,011,197

U.S. Pat. No. 6,096,555

U.S. Pat. No. 6,107,543

Van Etten W J, Steen R G, Nguyen H, Castle A B, Slonim D K, Ge B, Nusbaum C, Schuler G D, Lander E S & Hudson T J (1999) Radiation Hybrid Map of the Mouse Genome. Nat Genet22:384-387.

Wakayama T, Perry A C, Zuccotti M, Johnson K R & Yanagimachi R (1998) Full-Term Development of Mice from Enucleated Oocytes Injected with Cumulus Cell Nuclei. Nature 394:369-374.

Wakayama T, Rodriguez I, Perry A C, Yanagimachi R & Mombaerts P (1999) Mice Cloned from Embryonic Stem Cells. Proc Natl Acad Sci U S A 96:14984-14989.

Wakayama T, Shinkai Y, Tamashiro K L, Niida H, Blanchard D C, Blanchard R J, Ogura A, Tanemura K, Tachibana M, Perry A C, Colgan D F, Mombaerts P & Yanagimachi R (2000) Cloning of Mice to Six Generations. Nature 407:318-319.

Wells C & Brown S D (2000) Genomics Meets Genetics: Towards a Mutant Map of the Mouse. Mamm Genome 11:472-477.

Williams R, Gu J, Qi S & Lu L (2001) The Genetic Structure of Recombinant Inbred Mice: High Resolution Consensus Maps for Complex Trait Analysis. Genome Biology 2:0046.1-0046.18.

Wilmut I, Schnieke A E, McWhir J, Kind A J & Campbell K H (1997) Viable Offspring Derived from Fetal and Adult Mammalian Cells. Nature 385:810-813.

WO 9,858,050

It will be understood that various details of the invention can be changed without departing from the scope of the invention. Furthermore, the foregoing description is for the purpose of illustration only, and not for the purpose of limitation—the invention being defined by the claims.

What is claimed is:

1. A method for identifying a genetic locus that modulates a phenotype, the method comprising:
(a) providing a renewable population of genetically diverse individuals; and
(b) mapping the genomes of individuals within the renewable population of genetically diverse individuals that display the phenotype, whereby a genetic locus that modulates the phenotype is identified.

2. The method of claim 1, wherein the renewable population of genetically diverse individuals comprises:
(a) individuals produced by intercrossing recombinant inbred lines;
(b) individuals produced by backcrossing recombinant inbred lines;
(c) a cloned population of genetically diverse individuals; or
(d) a panel of cell lines derived from genetically diverse individuals.

3. The method of claim 1, wherein an individual of the renewable population of genetically diverse individuals comprises a diploid, tetraploid, or polyploid organism, or a cell derived there from.

4. The method of claim 3, wherein the organism is selected from a group consisting of an animal and a plant.

5. The method of claim 4, wherein the animal is a mammal.

6. The method of claim 5, wherein the mammal is a rodent.

7. The method of claim 6, wherein the rodent is a mouse.

8. The method of claim 2, wherein the recombinant inbred lines comprise less than about 500 lines.

9. The method of claim 8, wherein the recombinant inbred lines comprise less than about 100 lines.

10. The method of claim 2, wherein the recombinant inbred lines comprise one or more recombinant inbred lines selected from the group consisting of mouse lines AXB, BXA, CXB, and BXD.

11. The method of claim 2, wherein the recombinant inbred lines comprise recombinant inbred lines derived from at least 3 non-recombinant parent lines.

12. The method of claim 11, wherein the recombinant inbred lines comprise recombinant inbred lines derived from at least 4 non-recombinant parent lines.

13. The method of claim 12, wherein the recombinant inbred lines comprise recombinant inbred lines derived from at least 8 non-recombinant parent lines.

14. The method of claim 11, wherein the at least three non-recombinant parent lines comprise one or more non-recombinant parent lines selected from the group consisting of mouse lines C57BL/6, BALB/c, C3H, A, 129, and DBA/2.

15. The method of claim 2, wherein the cloned population or the panel of cell lines is derived from recombinant inbred line intercrosses, recombinant inbred line backcrosses, an F2 population, or a natural population.

16. The method of claim 15, wherein the recombinant inbred lines comprise recombinant inbred lines derived from at least 3 non-recombinant parent lines.

17. The method of claim 16, wherein the recombinant inbred lines comprise recombinant inbred lines derived from at least 4 non-recombinant parent lines.

18. The method of claim 17, wherein the recombinant inbred lines comprise recombinant inbred lines derived from at least 8 non-recombinant parent lines.

19. The method of claim 1, wherein the mapping comprises analysis of genetic polymorphisms segregating in the renewable population of genetically diverse individuals.

20. The method of claim 1, wherein the phenotype is selected from the group consisting of a visible phenotype, a physiological phenotype, a behavioral phenotype, a susceptibility phenotype, a cellular phenotype, a molecular phenotype, and combinations thereof.

21. The method of claim 20, wherein the molecular phenotype is selected from the group consisting of a level of gene expression, a splice selection, a level of protein, a protein type, a protein modification, a level of lipid, a lipid type, a lipid modification, a level of carbohydrate, a carbohydrate type, a carbohydrate modification, and combinations thereof.

22. The method of claim 1, wherein the phenotype is modulated by a non-genetic factor.

23. The method of claim 22, wherein the phenotype is modulated by an interaction between two or more non-genetic factors.

24. The method of claim 22, wherein the non-genetic factor is an environmental condition or drug exposure.

25. The method of claim 1, wherein the phenotype is modulated by an interaction between a genetic locus and a non-genetic factor.

26. The method of claim 25, wherein the non-genetic factor is an environmental condition or drug exposure.

27. The method of claim 1, further comprising identifying two or more genetic loci that modulate the phenotype.

28. A method for producing a renewable population of genetically diverse individuals, the method comprising:
(a) intercrossing recombinant inbred lines;
(b) backcrossing recombinant inbred lines;
(c) cloning a population of genetically diverse individuals; or
(d) generating a panel of cell lines derived from genetically diverse individuals.

29. The method of claim 28, wherein an individual of the renewable population of genetically diverse individuals comprises a diploid, tetraploid, or polyploid organism, or a cell derived there from.

30. The method of claim 29, wherein the organism is selected from a group consisting of an animal and a plant.

31. The method of claim 30, wherein the animal is a mammal.

32. The method of claim 31, wherein the mammal is a rodent.

33. The method of claim 32, wherein the rodent is a mouse.

34. The method of claim 28, wherein the recombinant inbred lines comprise less than about 500 lines.

35. The method of claim 34, wherein the recombinant inbred lines comprise less than about 100 lines.

36. The method of claim 28, wherein the recombinant inbred lines comprise one or more recombinant inbred lines selected from the group consisting of mouse lines AXB, BXA, CXB, and BXD.

37. The method of claim 28, wherein the recombinant inbred lines comprise recombinant inbred lines derived from at least 3 non-recombinant parent lines.

38. The method of claim 37, wherein the recombinant inbred lines comprise recombinant inbred lines derived from at least 4 non-recombinant parent lines.

39. The method of claim 38, wherein the recombinant inbred lines comprise recombinant inbred lines derived from at least 8 non-recombinant parent lines.

40. The method of claim 37, wherein the at least three non-recombinant parent lines comprise one or more non-recombinant parent lines selected from the group consisting of mouse lines C57BL/6, BALB/c, C3H, A, 129, and DBA/2.

41. The method of claim 28, wherein the cloned population or the panel of cell lines is derived from recombinant inbred line intercrosses, recombinant inbred line backcrosses, an F2 population, or a natural population.

42. The method of claim 41, wherein the recombinant inbred lines comprise recombinant inbred lines derived from at least 3 non-recombinant parent lines.

43. The method of claim 42, wherein the recombinant inbred lines comprise recombinant inbred lines derived from at least 4 non-recombinant parent lines.

44. The method of claim 43, wherein the recombinant inbred lines comprise recombinant inbred lines derived from at least 8 non-recombinant parent lines.

45. A renewable population of genetically diverse individuals produced by the method of claim 28.

46. A method for identifying an interaction between a genetic locus and a non-genetic factor, wherein the interaction modulates a phenotype, the method comprising:
(a) providing a renewable population of genetically diverse individuals;
(b) providing a non-genetic factor to the renewable population; and
(c) mapping the genomes of individuals that display the phenotype, whereby an interaction between a genetic locus and the non-genetic factor that modulates a phenotype is identified.

47. The method of claim 46, wherein the renewable population of genetically diverse individuals comprises:
(a) individuals produced by intercrossing recombinant inbred lines;
(b) individuals produced by backcrossing recombinant inbred lines;
(c) a cloned population of genetically diverse individuals; or
(d) a panel of cell lines derived from genetically diverse individuals.

48. The method of claim 46, wherein the phenotype is selected from the group consisting of a visible phenotype, a physiological phenotype, a behavioral phenotype, a susceptibility phenotype, a cellular phenotype, a molecular phenotype, and combinations thereof.

49. The method of claim 48, wherein the molecular phenotype is selected from the group consisting of a level of gene expression, a splice selection, a level of protein, a protein type, a protein modification, a level of lipid, a lipid type, a lipid modification, a level of carbohydrate, a carbohydrate type, a carbohydrate modification, and combinations thereof.

50. The method of claim 46, further comprising identifying an interaction among two or more genetic loci and a non-genetic factor.

51. The method of claim 46, further comprising identifying an interaction among a genetic locus and two or more non-genetic factors.

52. The method of claim 46, wherein the non-genetic factor is an environmental condition or drug exposure.

53. The method of claim 46, further comprising identifying an interaction among two or more genetic loci and two or more non-genetic factors.

54. A method for identifying an epistatic interaction between genetic loci that modulate a phenotype, the method comprising:
(a) providing a first renewable population of genetically diverse individuals;
(b) identifying individuals of the first renewable population that display a phenotype;
(c) mapping the genomes of the individuals of (b), whereby a first genetic locus that modulates the phenotype is identified;
(d) establishing a second renewable population of genetically diverse individuals wherein the first genetic locus identified in (c) is held constant;
(e) identifying individuals among the second renewable population of genetically diverse individuals that display the phenotype; and
(f) mapping the genomes of the individuals of (e), whereby a second genetic locus that epistatically interacts with the first genetic locus to modulate the phenotype is identified.

55. The method of claim 54, further comprising identifying an epistatic interaction between gene networks.

56. A method for producing recombinant inbred lines, comprising:
(a) intercrossing at least three non-recombinant inbred parent lines to produce recombinant hybrids;
(b) intercrossing the recombinant hybrids one or more generations to produce genetically diverse recombinant individuals; and
(c) inbreeding each recombinant individual to produce a recombinant inbred line.

57. The method of claim 56, wherein the recombinant inbred lines comprise recombinant inbred lines derived from at least 4 non-recombinant parent lines.

58. The method of claim 57, wherein the recombinant inbred lines comprise recombinant inbred lines derived from at least 8 non-recombinant parent lines.

59. A recombinant inbred line produced by the method of claim 56.
